(19) United States
(12) Patent Application Publication
(10) Pub. No.: US 2013/0336953 A1
(43) Pub. Date: Dec. 19, 2013

(54) USE OF CREATINE KINASE FOR PREVENTING OR TREATING ADDICTION TO OPIATES

(71) Applicants: Yannick Goumon, Bischheim (FR); Alexis Laux - Biehlmann, Strasbourg (FR); Denise Stuber, Eckbolsheim (FR)

(72) Inventors: Yannick Goumon, Bischheim (FR); Alexis Laux - Biehlmann, Strasbourg (FR); Denise Stuber, Eckbolsheim (FR)

(73) Assignees: UNIVERSITÉ DE STRABOURG, Strabourg (FR); CENTRE NATIONAL DE LA RECHERCHE SCIENTIFIQUE - CNRS, Paris Cedex 16 (FR)

(21) Appl. No.: 13/984,098

(22) PCT Filed: Feb. 3, 2012
(86) PCT No.: PCT/FR2012/050245

§ 371 Date: Aug. 29, 2013

(30) Foreign Application Priority Data
Feb. 8, 2011 (FR) 1151000
Jul. 29, 2011 (FR) 1156996

(51) Int. Cl. A61K 38/45

(52) U.S. Cl. CPC A61K 38/45
USPC 424/94.5; 435/194; 435/17; 435/283.1

(57) ABSTRACT

The present invention relates to creatine kinase for use as a medicinal product or in the prevention or treatment of addictions or of addictive disorders associated with opiates, and to various other analytical uses or for filtration of creatine kinase.

### FIELD OF TECHNOLOGY

The present invention relates to the use of creatine kinase for obtaining a medicinal product intended for preventing or treating an addiction or an addictive disorder.

The present invention notably finds application in the area of addictive disorders associated with an increase in the level of morphine and derivatives thereof, in particular the level of endogenous morphine and derivatives thereof.

In the following description, the references in brackets ([]) refer to the list of references given at the end of the text.

### PRIOR ART

Creatine kinase (CK, EC 2.7.3.2), also called phosphocreatine kinase or creatine phosphokinase (CPK), is an enzyme expressed by various tissues including the brain and muscles, where it is present in cytoplasmic and mitochondrial form. There are several isoenzymes (present in the form of noncovalent dimers): CK-MM present in muscles, CK-MB found in the myocardial cells, and CK-BB which occurs in the brain. Mitochondrial CK (mtCK) also occurs. This enzyme catalyzes the reversible conversion of creatine to phosphocreatine via the use of adenosine triphosphate (ATP, FIG. 1). This enzyme is essential for the production of ATP from stored phosphocreatine. Phosphocreatine, via ATP, thus constitutes a reservoir of energy that is rapidly usable for the muscles and other organs including the brain. CK (isoforms MM and MB; dimers) is released in the blood during tissue lesions causing cellular lysis (for example in a muscle disorder such as rhabdomyolysis, muscular exertion, myocardial infarction, etc.). Assaying isoenzymes in the blood makes it possible to differentiate the origin of the cellular destruction. Thus, increase in blood CK-MB has been used in a blood test for diagnosing myocardial infarction.

The alkaloids are basic nitrogen-containing heterocyclic organic molecules, of natural origin, which can have pharmacologic activity. Although many alkaloids are toxic (such as strychnine or aconitine), some are used in medicine for example for their analgesic properties (such as morphine, codeine), in sedation protocols (anesthesia) often accompanied by hypnotics, or as antimalarial agent (quinine, chloroquinine) or anticancer agent (taxol, vinblastine, vincristine).

The opiates are chemical molecules that act by binding to the opioid receptors Mu, Delta or Kappa, which are expressed in the central and peripheral nervous system and the gastrointestinal tract. These receptors mediate both the beneficial effects and the side effects of the opiates (alkaloids) and opioids (peptides). The opiates are among the oldest drugs known. The analgesic (pain-relieving) effects of the opiates are due to reduced perception of pain, reduced reaction to pain, and increased tolerance to pain. The main side effects of the opiates are sedation, respiratory depression, and constipation. The opiates can cause suppression of cough, which can be both an indication for administration of opiates or an unexpected side effect. Psychological and physical dependence can develop with the continuous administration of opiates, which leads to a withdrawal syndrome if they are stopped suddenly. The opiates are well known for their capacity to produce a feeling of euphoria, thus motivating some people to use them for recreational purposes. There are five broad classes of opiates: (A) the natural opiates are alkaloids contained in the resin of the opium poppy (Papaver somniferum) but also in other living organisms (mammals and invertebrates), namely mainly morphine and morphine derivatives, for example codeine and thebaine, but not papaverine and noscapine, which have a different mechanism of action. The following elements could be regarded as natural opiates: the leaves of Mitragyna speciosa (also called Kratom) contain some natural opiates, acting via the Mu and Delta receptors. Salvinorin A, found naturally in the plant Salvia divinorum, is an agonist of the kappa opioid receptors; (B) the semi-synthetic opiates created starting from natural opiates such as hydromorphone, hydrocodone, oxycodone, oxymorphone, desomorphine, nicomorphine, dipropanoylmorphine, benzylmorphine, ethylmorphine and buprenorphine; (C) the completely synthetic opiates such as fentanyl, pethidine, methadone, tramadol and dextropropoxyphene; (D) the endogenous peptide opioids produced naturally in the body (such as endorphins, enkephalins, dynorphins, and endomorphins); and (E) their synthetic or semi-synthetic derivatives (DDPE, etc.).

It is known from the prior art that chronic administration of alkaloids or of other drugs of abuse such as alcohol, nicotine, etc. leads to tolerance or even dependence (addiction) of the human or animal organism.

Morphine is a natural opiate alkaloid used as medication against pain (analgesic). The main alkaloid obtained from the opium poppy (Papaver somniferum), morphine is considered as the reference against which all the other analgesics are compared in terms of efficacy. It is most often used in the form of a salt, sulfate or hydrochloride, with identical efficacies. To date, morphine is the most effective analgesic and the one most commonly used for relieving various types of physical pain. However, its use poses many problems owing to the dependence that it can induce. Thus, it is listed internationally (CII) as a narcotic. Endogenous morphine (EM), structurally identical to the morphine from the opium poppy, as well as several derivatives thereof have been characterized in various mammalian tissues and cells (chromaffin cells and polymorphonuclear neutrophils) [Herbert et al., Nat. Prod. Rep., 17: 317-322, 2000; Stefano et al., Trends Neurosci., 23: 436-442, 2000; Zhu et al., J. Immunol., 175(11): 7357-7362, 2005; Glattard et al., PLoS One, 5(1): e8791, 2010] [1, 2, 24, 25], and are present, for example, in the secretory vesicles of certain neurons [Bianchi et al., Brain Res., 627: 210-215, 1993; Bianchi et al., Adv. Neuroimmunol., 4: 83-92, 1994; Guarna et al., J. Neurochem., 70: 147-152, 1998; Muller et al., PLoS One, 3: Epub www.plosone.org/doi/pone.0001641, 2008] [3-6]. Endogenous morphine is also found in various biological fluids including cerebrospinal fluid, blood and urine. Just as in plants, the synthesis route of morphine in mammals is derived from that of dopamine (FIG. 2) [Boettcher et al., Proc. Natl. Acad. Sci. USA, 102: 8495-8500, 2005; Goumon et al., An. R. Acad. Nac. Farm., 75: 389-418, 2009] [7, 8]. The catabolism of morphine (endogenous and exogenous) by the UDP-glucuronosyl transferase (UGT) superfamily leads to the formation of morphine-3-glucuronide (M3G, devoid of analgesic activity) and morphine-6-glucuronide (M6G, which has analgesic activity greater than that of morphine, 1 to 600 times) [Lotsch and Geisslinger, Clin. Pharmacokinet., 40: 485-499, 2001] [9]. However, other endogenous derivatives are also found: normorphine, nocodeine-3-6-diglucuronide, hydrocodone, hydromorphone, etc. A correlation was established between the serum morphine level and sepsis (severe systemic infection), which indicates that endogenous morphine represents a serum biomarker of infections [European patent application No. 07301692.5] [10]. Moreover, recent results indicate that immunoreactivity related to morphine is distributed throughout the mouse brain and with a higher density of labeling localized to the cerebral regions involved in motor activity and memory [Laux et al., J. Comp. Neurol., 519(12): 2390-416, 2011; Charron et al., Brain, PMID: 21742735, 2011] [11, 12].

Furthermore, it is known from the prior art that several disorders are associated with an increase in the levels of endogenous morphine or derivatives thereof relative to the normal level. These are for example Parkinson's disease, anorexia, bulimia, alcoholism, schizophrenia, septic infections, surgical stress, shingles, or stress associated with lipopolysaccharide (LPS) [Arun et al., Indian J. Med. Res., 107: 231-238, 1998; Glattard et al., PLoS One, 1: e8791, 2010; Goumon et al., Neurosci. Lett., 293: 135-138, 2000; Lee and Spector, J. Pharmacol. Exp. Ther., 257: 647-650, 1991; Madbouly et al., Br. J. Surg., 97: 759-764, 2010; Marrazzi et al., Life Sci., 60: 1741-1747, 1997] [20-25].

Regarding a potential therapeutic link with a decrease in the levels of endogenous morphine, various studies have shown that the use of antagonists of the opioid Mu receptors inhibiting the effects of endogenous morphine (naloxone, naltrexone) is beneficial with respect to cutaneous pruritus, obesity, sexual problems, alcoholism, schizophrenia and Parkinson's disease [Batki et al., Am. J. Addict., 16(4): 253-259, 2007; Green et al., J. Subst. Abuse Treat., 34(1): 61-71, 2008; Goodman et al., ChemMedChem., 2(11): 1552-1570, 2007; Meyer, J. Clin. Psychopharmacol., 28(6): 722-723, 2008] [26-29].

In the hospital environment, blocking of the action of the opiates (codeine, morphine, heroin, oxycodone, etc.) during withdrawal, overdose or addiction is accomplished by injecting nonspecific antagonists (i.e. naloxone, naltrexone, etc.) of the opioid receptors (Mu, Delta, Kappa). This approach has the drawback that it blocks all types of opioid receptors but also the action of the alkaloids and of the endogenous opioids (i.e. peptides including β-endorphin, endomorphins, etc.) on their receptors, which causes many side effects and in particular hyperalgesia, pulmonary edemas and cardiac arrhythmias [Van Dorp et al., Expert Opin. Drug Saf., 6(2): 125-32, 2007] [19]. The ideal would therefore be to identify molecules capable of binding and blocking specifically the action of the opiates without acting on their receptors.

The binding of morphine or derivatives thereof to proteins has already been described [Misra et al., Nature, 232: 48-50, 1971; Mullis et al., J. Pharmacol. Exp. Ther., 208: 228-231, 1979; Nagamatsu et al., Drug Metab. Dispos., 11: 190-194, 1983; Olsen et al., Clin. Pharmacol. Ther., 17(6): 677-684, 1975] [19-22]. In fact, morphine has been characterized as binding weakly to serum albumin, as well as acidic alpha-1-glycoprotein [Leow et al., Ther. Drug Monit., 15(5): 440-447, 1993] [23], and it has recently been described that the phosphatidylethanolamine-binding protein (PEBP) is capable of binding only M6G and M3G at low affinity, but does not have any affinity for morphine [Atmanene et al., Med. Sci. Monit., 15: BR178-187, 2009; Goumon et al., J. Biol. Chem., 281(12): 8082-9, 2006] [13, 16]. Other studies have indicated that a protein of 50-53 kDa can spontaneously bind radioactive morphine covalently (protein not characterized, without biochemical proof) [Nagamatsu and Hasegawa, Biochem. Pharmacol., 43: 2631-2635, 1992] [14]. It has also been described that this morphine-protein bond was diminished in the presence of selenium [Nagamatsu and Hasegawa, Drug Chem. Toxicol., 16: 241-253, 1993] [15].

There is therefore a real need to identify molecules for binding and blocking specifically the action of the opiates, notably morphine and derivatives thereof, without going via blocking of the opioid receptors, overcoming the defects, drawbacks and obstacles of the prior art, and thus improving the management of withdrawal, overdose or addictions or addictive disorders, notably associated with opiates, while reducing their costs.

### DESCRIPTION OF THE INVENTION

The inventors were the first to observe that although strong immunolabeling is found in tissues, suggesting the presence of large amounts of endogenous morphine, only traces of opioids are found in the tissues and fluids when said opioids are extracted by means of protocols using protein precipitation steps. Based on this observation, they put forward the hypothesis that endogenous morphine, but also exogenous morphine, could a priori form complexes with proteins in the blood and the tissues.

The inventors then identified, quite unexpectedly, alkaloid-creatine kinase B complexes (CK-BB) of very high affinity, noncovalent. These complexes, which can be detected by Western blotting in special conditions using an anti-morphine antibody, are resistant to detergents, reducing agents and heat treatments. Treatments with naloxone, naltrexone, as well as urea followed by heating, dissociate these opiate-creatine kinase B complexes. In addition they demonstrated that similar complexes can be formed between alkaloids and creatine kinase M (muscular).

The present invention therefore relates to a creatine kinase or a derivative thereof for use in the prevention or treatment of an addiction or of an addictive disorder, and dysfunctions resulting from said addiction or addictive disorder, or from disorders having raised levels of endogenous morphine.

Thus, the present invention relates to the use of a creatine kinase or of a derivative thereof as medicinal product or for preparing a medicinal product intended for preventing or treating an addiction or an addictive disorder, and dysfunctions resulting from said addiction or addictive disorder, or from disorders having raised levels of endogenous morphine.

According to the invention, it is in particular an addiction or an addictive disorder associated with opiates, preferably with the endogenous or exogenous natural opiates from opium, more preferably with morphine and derivatives thereof.

“Creatine kinase” means, in the sense of the present invention, the CK-MM present in the muscles, the CK-MB found in the myocardial cells, the CK-BB that occurs in the brain and the mtCK (sarcomere and ubiquitous) which is present in the mitochondria of mammalian cells, as well as the monomers of CK (CK-B and CK-M).

“Derivatives of creatine kinase” means, in the sense of the present invention, fragments of creatine kinase or any fragment having an identical, similar or modified amino acid sequence (e.g. “retro inverso” resistant to proteolysis) and preserving the capacity for binding the opiates. For example, it can be C-terminal fragments of creatine kinase preserving the capacity for binding the opiates.

“Addiction” means, in the sense of the present invention, a chronic disorder that develops progressively, characterized by a harmful attachment to an addictive substance such as alcohol, nicotine, the alkaloids, etc.

“Addictive disorder” means, in the sense of the present invention, any disorder associated with an increase in the level of addictive substance such as alcohol, nicotine, the alkaloids, etc., relative to the normal level. In particular, it can be an increase in the level of endogenous morphine and/or of derivatives thereof relative to the normal level, for example in Parkinson's disease, anorexia, bulimia, alcoholism, schizophrenia, septic infections, surgical stress, or stress associated with lipopolysaccharide (LPS) and all other disorders involving the endogenous and/or exogenous opiates.

“Opiates” means, in the sense of the present invention, endogenous or exogenous chemical molecules that act by binding to the opioid receptors, which are mainly located in the central and peripheral nervous system and the gastrointestinal tract. For example, they are the natural (exogenous) alkaloids of opium such as morphine and derivatives thereof (e.g. codeine, thebaine), semi-synthetic opiates created starting from natural opiates (e.g. hydromorphone, desomorphine, nicomorphine, dipropanoylmorphine, benzylmorphine, ethylmorphine, buprenorphine), completely synthetic opiates (e.g. fentanyl, pethidine, methadone, tramadol, dextropropoxyphene), endogenous peptide opioids produced naturally in the body (e.g. endorphins, enkephalins, dynorphins, endomorphins), as well as their synthetic or semi-synthetic peptide derivatives.

The present invention further relates to a creatine kinase or a derivative thereof as medicinal product for capturing opiates, preferably the natural endogenous or exogenous opiates from opium, more preferably morphine and derivatives thereof, in human or animal tissues (e.g. cerebral, hepatic, pulmonary) or body fluids, and thus makes it possible to decrease their circulating level.

The medicinal product can be in any form that can be administered to an animal or to a human. Administration can be direct, i.e. in pure or almost pure form, or after mixing the active principle (creatine kinase or a derivative thereof) with a pharmaceutically acceptable vehicle and/or medium. The medicinal product can for example be intended for administration by the oral route (liquid formulation e.g. solution, syrup, suspension, emulsion, drops; effervescent form e.g. tablet, granules, powder; oral powder or multiparticulate system e.g. beads, granules, mini-tablets and microgranules; orodispersible form e.g. orodispersible tablet, lyophilized cachet, thin film, tablet to be crunched, tablet and capsule, medical chewing gum), administration by the buccal or sublingual route (e.g. buccal or sublingual tablet, muco-adhesive preparation, pastille, oro-mucosal drops, spray), or parenteral administration (e.g. injectable solution).

The present invention further relates to a pharmaceutical composition for use in the prevention or treatment of an addiction or of an addictive disorder, comprising a creatine kinase or a derivative thereof as active principle and a pharmaceutically acceptable vehicle. In particular it can be an addiction or an addictive disorder associated with opiates, preferably with the natural alkaloids from opium, more preferably with morphine and derivatives thereof, or with the natural endogenous alkaloids, preferably endogenous morphine and derivatives thereof.

The present invention further relates to a method of analysis of opiates in a sample comprising:

a) contacting said sample with a creatine kinase or a derivative thereof; and

b) detecting any complexes formed between said creatine kinase or said derivative thereof and at least one opiate present in the sample.

According to a particular embodiment, said method of analysis can further comprise a step c) in which the results of detection obtained in step b) are compared relative to a negative and/or positive control.

“Positive control” and “negative control” mean, in the sense of the present invention, a control sample respectively comprising or not comprising at least one opiate. This control makes it possible to compare the result of detection obtained in step b) of the method of the invention and to detect a false positive or false negative, as applicable.

According to a particular embodiment, said method of analysis can further comprise a step c_{bis}) in which the results of detection obtained in step b) are compared with a measurement standard, which permits qualitative and/or quantitative analysis of the opiates.

“Measurement standard” means, in the sense of the present invention, one or more analysis (analyses) carried out on solutions manufactured for the needs of the analysis according to the invention comprising a known content of opiate(s). Based on the results of analysis obtained on these manufactured solutions, it is possible to determine the presence of opiate(s) in the sample and even its(their) concentration by comparison, for example based on a standard curve constructed for the measurement standards.

According to the invention, steps c) and c_{bis}) can both be carried out before, during or after steps a) and b) of the method of the invention. According to the invention, steps c) and c_{bis}) can be carried out simultaneously or successively, including with steps a) and b), for example on a multiwell plate for simultaneously or successively carrying out several analyses, including standard measurements.

The methods of analysis of opiates according to the present invention can be used for analyzing for example the endogenous or exogenous natural alkaloids, for example morphine as well as derivatives thereof. For this purpose, creatine kinase or a derivative thereof can be labeled, for example by fluorescent, radioactive, etc. labeling. Detection is then carried out by any suitable type of system known from the prior art.

The present invention further relates to a kit for analysis of opiates comprising a creatine kinase or a derivative thereof and means for detecting the complexes formed between said creatine kinase or said derivative thereof and at least one opiate. The detecting means are for example the aforementioned markers.

The present invention further relates to the use of a creatine kinase or a derivative thereof in a method of filtration of a body fluid for capturing opiates.

“Body fluid” means, in the sense of the present invention, for example saliva, urine, blood, cerebrospinal fluid, etc.

The present invention further relates to a device for capturing opiates in the presence of a creatine kinase or of a derivative thereof, in tissues (e.g. brain, liver, lung biopsy) or body fluids (e.g. saliva, urine, blood, cerebrospinal fluid).

According to a particular embodiment of the invention, the device can consist of a system for dialysis of a body fluid, preferably for renal, hepatic, peritoneal dialysis, dialysis of the blood or hemodialysis. For example said dialysis system can comprise a first circuit comprising creatine kinase, which comes into contact with the body fluid to be treated through one or more membranes or filters for recovering the creatine kinase once it is complexed with opiates. The second circuit comprises a system for decomplexing the creatine kinase before it is put back in circulation in the first circuit. Another example of dialysis system can comprise a circuit with a membrane or a filter identical to that used above in which the body fluid to be treated circulates. The other side of the membrane or filter is traversed by a solution of creatine kinase circulating in countercurrent and which is eliminated after passing through the membrane or filter.

This device can be used for example in the detoxification of body fluids, for example for treatment of a patient comprising high doses of opiate(s).

Other advantages may become apparent to a person skilled in the art on reading the experimental results obtained on the basis of the protocols described in the following examples carried out by the inventors, and illustrated in a nonlimiting manner by the accompanying drawings.

### BRIEF DESCRIPTION OF THE DRAWINGS

FIG. 1 shows the enzymatic reaction catalyzed by creatine kinase.

FIG. 2 shows the putative synthesis route of endogenous morphine in mammals according to [3].

FIG. 3 shows (A) the immunoreactivity observed for the alkaloids in mouse brain extracts using 5 different anti-morphine antibodies by Western blotting, (B) the effects of different buffers and heat treatments on dissociation of the “morphine-type molecules/protein” complex by Western blotting, (C) disappearance of immunoreactivity for the alkaloids after treatment of the transfer membranes with a buffer containing urea and heat treatment, (D) replacement of the immunoreactive alkaloid bound to the 42 kDa protein complexed with the morphine-type molecules by naloxone.

FIG. 4 shows (A) immunodetection of the protein complexed with the morphine-type molecules after two-dimensional gel, (B) localization of the proteins binding the alkaloids to the corresponding two-dimensional gel stained with silver nitrate (rectangles), (C) effect of urea and of temperature on creatine kinase BB and immunoreactivity for the alkaloids and human creatine kinase BB, (D) effect of naloxone on human creatine kinase BB.

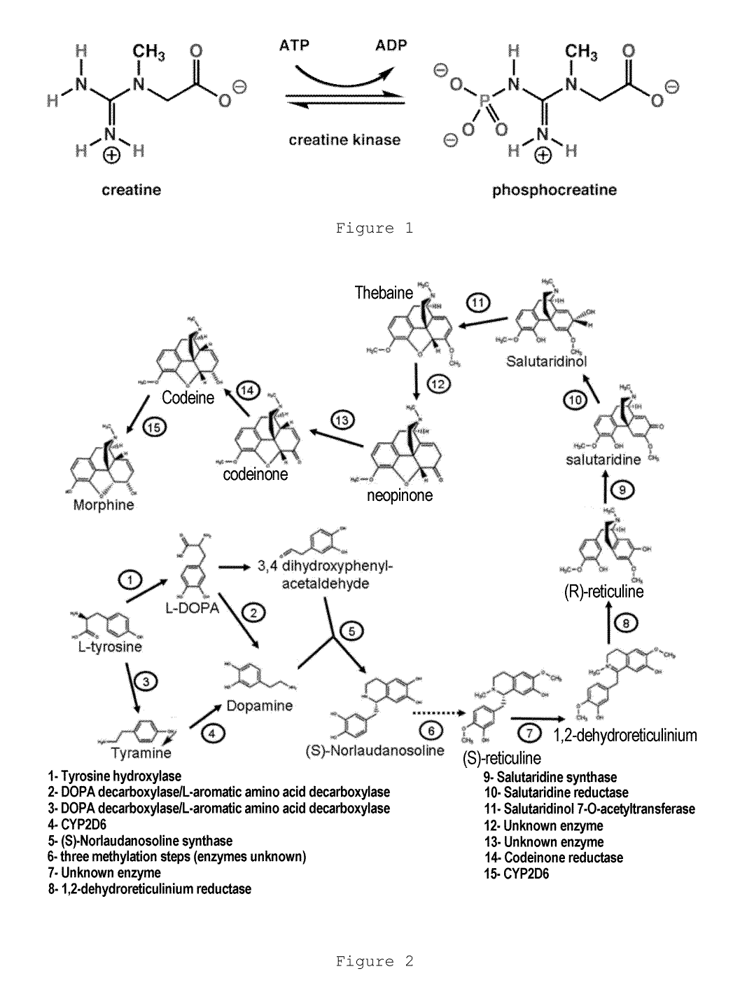

### EXAMPLES

### Example 1: Materials And Methods

### Antibodies

The monoclonal murine anti-morphine antibodies 6D6 and 3A6 (Aviva System Biology, ref: 16002 and AMM00033 respectively, San Diego, USA) were produced using a ((5-alpha-6-alpha)-7-8-didehydro-4-5-epoxy-17methylmorphinan-3-5-diol)-BSA conjugate.

The polyclonal sheep antibody ABD6330-0004 was obtained with respect to 3-(O-carboxymethyl)morphine conjugated with limpet hemocyanin (AbD Serotec, Oxford, UK). The polyclonal sheep antibody ABD6330-0014 was obtained after immunization with morphine conjugated with limpet hemocyanin.

The goat anti-morphine antibody G61 was supplied by Prof. R. Hain (Cardiff School of Medicine, University Hospital of Wales, Cardiff, UK).

The polyclonal rabbit antibody directed against creatine kinase B (CK-BB; ABCAM92452) was obtained with respect to the C-terminal portion of human CK-BB.

### Western Blot

The proteins were separated on SDS-PAGE gels (10% acrylamide Criterion XT; migration buffer XT-MOPS; BioRad, Marnes-la-Coquette, France). The samples were suspended in 15 μl of different loading buffer TPx:

Loading buffer 1 (TP1): 30 mM Tris HCl pH 6.8, 0.5% SDS, 5 mM EDTA, 2% glycerol, 0.5% β-mercaptoethanol, and 0.05% of bromophenol blue.

Loading buffer 2 (TP2): 125 mM Tris HCl pH 6.8, 2% SDS, 5 mM EDTA, 5% glycerol, 50 mM DTT, and 0.05% of bromophenol blue.

Loading buffer 3 (TP3): 10 mM Tris HCl pH 8, 3% SDS, 1 mM EDTA, 2.5% glycerol, 5 mM DTT, and 0.05% of bromophenol blue.

Loading buffer 4 (TP4, lithium dodecyl sulfate buffer): 250 mM Tris HCl pH 8.5, 2% lithium dodecyl sulfate buffer (LDS), 0.5 mM EDTA, 10% glycerol, 0.075% of Coomassie Blue G250, 0.025% of phenol red, reducing agent XT (BioRad).

Loading buffer 5 (TP5, urea buffer): 60 mM Tris HCl pH 6.8, 2% SDS, 4M urea, 5 mM EDTA, 5% glycerol, 1% β-mercaptoethanol.

The samples resuspended in the loading buffers were submitted (or not) to heat treatment (100° C.) for 0, 5, 10 or 15 minutes before being deposited on the gel. After migration, the proteins were electrotransferred onto polyvinyldifluorene membrane (GE Healthcare Bioscience) [Goumon et al., J. Biol. Chem., 281(12): 8082-8089, 2006] [16]. The membrane was treated for 30 minutes with a saturated solution (PBS supplemented with 3% of BSA and 0.05% of Tween 20). Then the primary antibodies were incubated for 1 h with the following dilution in PBS supplemented with 3% of BSA and 0.05% of Tween 20: (1) monoclonal murine antibody 6D6, diluted 1:4000; (2) monoclonal murine antibody 3A6, diluted 1:2000; (3) polyclonal sheep antibody ABD6330-0004, diluted 1:500; (4) polyclonal sheep antibody ABD6330-0014, diluted 1:500; (5) goat anti-morphine antibody G61, diluted 1:500; rabbit anti-CK-BB antibody, diluted 1:3000.

The immunoreactivity was detected using either donkey anti-mouse antiserum conjugated with HRP (P.A.R.I.S.; 1:50000 in the same buffer) or donkey anti-sheep antiserum conjugated with HRP (P.A.R.I.S.; 1:30000 in the same buffer) or donkey anti-goat antiserum conjugated with HRP (P.A.R.I.S.; 1:30000 in the same buffer) or donkey anti-rabbit antiserum conjugated with HRP (P.A.R.I.S.; 1:50000 in the same buffer). Kit Supersignal West Femto (Pierce, Rockford, USA) or substrate Luminata™ Forte Western HRP (Millipore, Molsheim, France). The nonspecific binding of each secondary antibody was tested by omitting the primary antibody, and no nonspecific labeling was shown.

### Measurements of Dissociation

Brain extracts were submitted to various treatments in order to observe the dissociation of the creatine kinase B—alkaloids complexes.

20 μg of brain extract or 2 μg of native human creatine kinase B (Fitzgerald 30-AC55) was incubated for 12h, at 37° C., in the presence of naloxone (ratio 1:1, m/m; Sigma Aldrich), in a final volume of 40 μl (diluted in water).

### Digestion in Gel and Analysis by Mass Spectrometry

The digestion in gel and the mass spectrometry analyses were carried out on a CapLC (Waters, http://www.waters.com) coupled to a hybrid orthogonal quadrupole tandem time-of-flight mass spectrometer (Q-TOF 2, Waters).

### Identification of the Proteins

The MS and MS/MS data were analyzed using a Mascot local server (MASCOT 2.0; Matrixscience, http://www.matrixscience.com) relative to a composite target-decoy database, including the UniProt protein sequences of Viridiplantae (554832 sequences; January 2009), of human keratins, of porcine trypsin and of all the corresponding reversed sequences (1109972 entries in total). The searches were performed with a mass tolerance of 250 ppm in MS mode and 0.4 Da in MS/MS mode for the nanoLC-MS/MS data. One failed cleavage per peptide was permitted and variable modifications were taken into account, such as carbamidomethylation of cysteine, oxidation of methionine and N-acetylation (N-ter protein).

No constraint of molecular weight of the protein or isoelectric point was applied. The Mascot results were loaded in MuDPIT mode in SCAFFOLD (Proteome software, http://www.proteomesoftware.com). The results were submitted to the following filtration criteria. For identifying proteins with two or more peptides, a Mascot ion score of more than 25 was required. In the case of single-peptide collisions, the score of the single peptide must be higher (minimum “difference score” of 0) than the Mascot significance threshold of 95%. Searching a target-decoy database makes it possible to monitor and estimate the level of identification of false positives of the study [Peng et al., J. Proteome Res., 2(1): 43-50, 2003; Elias and Gygi, Nat. Methods, 4(3): 207-214, 2007] [17, 18]. Thus, the final catalog of proteins has a level of false positives estimated at 1%.

### Example 2: Identification of “Morphine-Type Molecules/Protein” Complexes by Western Blot

### Immunodetection of “Morphine-Type Molecules/Protein” Complexes

In order to determine whether immunolabeling of the morphine type could be detected in the mouse brain, an immunoreactivity for morphine and/or derivatives thereof (M6G, M3G, codeine, normorphine and norcodeine) was tested in a mouse brain extract. For this purpose, brain extracts (10 μg) were resuspended in a loading buffer containing SDS and β-mercaptoethanol (control loading buffer, TP1). The samples that had not undergone a heat treatment (100° C., 10 minutes) were deposited on SDS-PAGE and the gel was transferred onto a PVDF membrane. Immunodetection was performed with 8 different antibodies (different with respect to host species, hapten, chemistry). These antibodies are not only specific for morphine but also have cross-reactions with some of its derivatives.

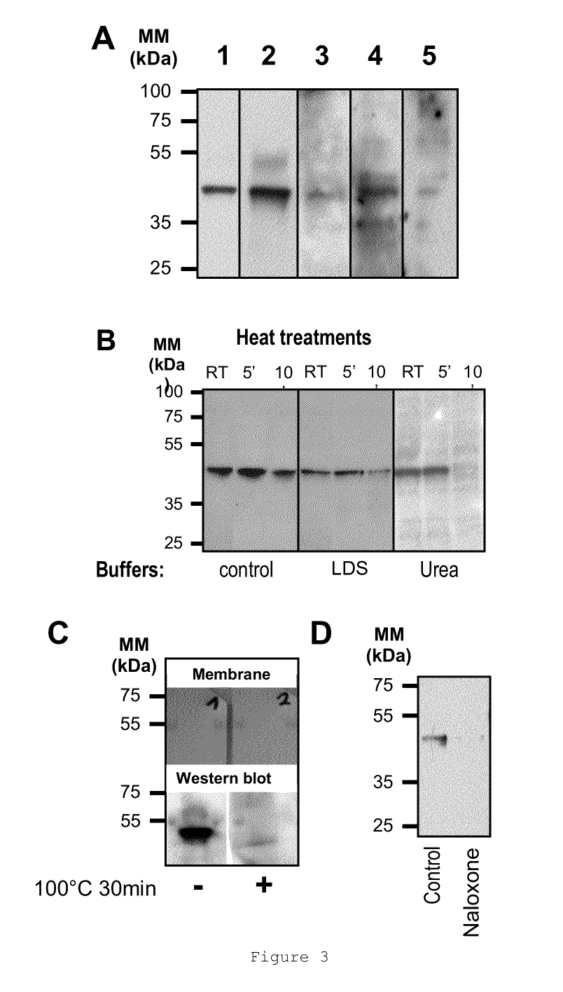

The results show that 5 anti-morphine antibodies among the 8 tested detect a single band at 40-45 kDa (tracks 1 to 5, FIG. 3A).

Taken together, these results suggest that the “morphine-type” labeling is present in the brain extracts, and may correspond to the alkaloids bound to a single brain protein. Moreover, these complexes are resistant to SDS and to β-mercaptoethanol (i.e. SDS-PAGE electrophoresis conditions), suggesting the presence of complexes with high affinity.

### Dissociation of the Complexes by Different Loading Buffers

In order to determine whether complexes with high affinity exist between the endogenous alkaloids and a protein, brain homogenates (20 μg) were deposited on polyacrylamide gels in the presence of different loading buffers (TP1, TP2, TP3, TP4, TP5) containing SDS or LDS, reducing agents (β-mercaptoethanol or DTT), or chaotropic agents (urea), at room temperature (RT) and in conditions of boiling (5 or 10 minutes at 100° C.). After transfer, the morphine-type labeling was demonstrated with the anti-morphine antibody 6D6 (Aviva Biosciences).

The results show that without heat treatment (RT, FIG. 3B), none of the loading buffers dissociated the complex (i.e. presence of a band at 40-45 kDa). After applying a heat treatment of 10 minutes (100° C.), a significant decrease in morphine-type immunolabeling was only observed for the loading buffer containing LDS (TP4), whereas the morphine-type labeling disappeared completely for the loading buffer containing urea (TP5).

Taken together, these results show that complexes with strong affinity involving a compound of the morphine type are only dissociated in the presence of loading buffer TP5 (urea) combined with a heat treatment. An interesting fact is that urea buffers are often used for dissociating protein-protein complexes. No dissociation is observed for buffers 2 and 3 (data not presented).

Additional experiments, carried out in parallel on a brain marker (chromogranin A, CGA) and silver-stained gels, did not show any decrease in immunoreactivity or total amount of proteins (data not presented). Moreover, the fact that the immunolabeling observed in the “control” condition (loading buffer TP1 containing neither LDS nor urea, FIG. 3B, left panel) did not show a significant decrease in morphine-type labeling confirms that loss of the labeling is not due to degradation of the proteins by the heat.

### Dissociation of the Complexes on Transfer Membrane

In order to determine whether labeling of this kind could be reversed after treatments on PVDF transfer membrane, an analysis by Western blot was carried out on a mouse brain extract (20 μg, loading buffer urea TP5 without heat treatment). The resulting gel was transferred onto a PVDF membrane, then the latter was incubated for 30 minutes at 100° C. in water or 2M urea supplemented with 0.5% of triton X100. After immunolabeling with the 6D6 antibody, the lines corresponding to incubation with water (RT and 100° C. for 30 minutes) do not show any decrease in morphine-type immunoreactivity (data not presented). An interesting fact is that whereas the track treated with the urea buffer at room temperature shows the presence of a band at 40-45 kDa (track “−”, FIG. 3C), the corresponding membrane submitted to the heat treatment shows complete disappearance of the labeling of the alkaloid (track “+”, FIG. 3C).

Taken together, these results show, as for the loading buffer, that the decrease in morphine-type labeling was induced by urea combined with heat treatment. This suggests that an alkaloid is bound with high affinity to a protein present in the mouse brain. The decrease in labeling is not associated with the heat treatment, since the labeling does not decrease for buffers not containing urea (data not presented).

### Measurements of Dissociation in Vitro

A recent study on mapping of the endogenous alkaloids in the mouse showed that the anti-morphine antibody 6D6 did not display cross-reaction with naloxone (antagonist of the Mu opioid receptor having a structure similar to that of morphine) in ELISA. On this basis, a competition test was performed on brain extracts in order to replace the morphine-type molecules with the antagonists not detected by the 6D6 antibody. For this purpose, the brain extracts were incubated with naloxone for 12 h at 37° C. The samples were deposited on a polyacrylamide gel (urea buffer without heat treatment) and membrane transfer was detected with the 6D6 antibody. The incubations with the antagonist (naloxone) significantly decreased the alkaloid labeling relative to the control experiments (same incubation carried out in the absence of antagonist, FIG. 3D).

Taken together, these results suggest that the morphine-type molecules are not bound covalently to a protein of the murine brain, and that these alkaloids can be replaced with a synthetic analog of morphine not immunodetected by the 6D6 antibody.

### Example 3: Characterization of the Protein Complexed with Morphine-Type Molecules

Proteomic Identification of the Protein Complexed with Morphine-Type Molecules

In order to identify the protein binding the endogenous morphine-type molecules, a proteomic approach was adopted. For this purpose, 125 μg of mouse brain extract was submitted to isoelectric focusing, and then separated on SDS-PAGE. Two experiments were carried out in parallel: (i) silver staining of the gel and (ii) transfer followed by Western blotting using the 6D6 antibody.

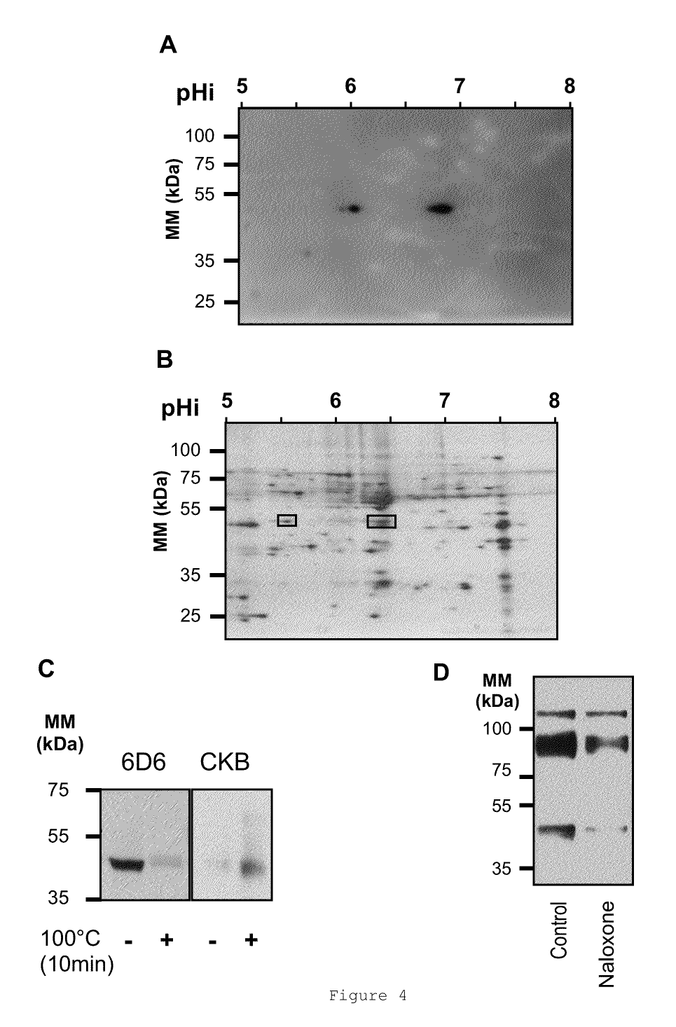

The results of Western blotting show the presence of two immunoreactive spots at 40-45 kDa and at pHi of 6.1 and 6.9 (FIG. 4A). The corresponding spots (FIG. 4B, cf. boxes) present in the silver-stained gel were digested with trypsin and analyzed by tandem mass spectrometry (or MS-MS), and revealed the presence of murine creatine kinase B in both spots (40 and 60% of coverage; 42 kDa).

The presence of two immunoreactive spots is probably due to the presence of a post-translational modification leading to a change of pHi.

These results indicate that the creatine kinase B is bound to the ligand of the endogenous morphine type.

### Dissociation of the “Morphine-Type Molecules/Human Creatine Kinase BB” Complexes

Commercial purified human brain creatine kinase BB (CK-BB, ref. 30-AC55, Fitzgerald Industrie International, active form) was tested for its morphine-type immunoreactivity using the 6D6 antibody. For this purpose, 2 μg of human creatine kinase BB was deposited on SDS-PAGE, then transferred onto membrane treated with the 6D6 antibody. The human brain creatine kinase BB displayed two immunoreactive bands with respect to the morphine-type molecules at 42 and 84 kDa corresponding to the monomer and dimer of creatine kinase BB, respectively (data not shown). An identical result was observed for CK-M (CK-M, ref. C3755, Sigma Aldrich; data not presented).

The dissociation of the morphine-type molecule of creatine kinase BB (deposit 0.5 μg on gel) treated in the presence of urea (TP5) whether or not combined with heat treatment (100° C., 10 minutes), was tested and immunodetected by Western blotting by means of the anti-morphine antibody 6D6. The results show that, as for CK-M (Sigma Aldrich, C3755) and the mouse brain extracts, the morphine-type immunolabeling is only lost when the complex has undergone the heat treatments combined with urea (track 6D6+vs−, FIG. 4C).

A parallel experiment using an antibody directed against the C-terminal portion of creatine kinase BB (ref. ab92452, ABCAM) was carried out on the same samples.

The results show that, surprisingly, the immunoreactivity of creatine kinase BB is low when the protein was only treated with urea (track CKB “−”, FIG. 4C), but increased after heat treatment (track CKB “+”, FIG. 4C), suggesting that the anti-creatine kinase BB antibody recognized a C-terminal epitope of creatine kinase B to which the morphine-type molecule was bound initially. The heat treatment in the presence of the urea buffer TP5 therefore permitted displacement of the alkaloid and thus left the epitope free for binding the anti-creatine kinase BB antibody. The same result was obtained when the experiment was carried out on a PVDF membrane treated with the urea buffer combined with heat treatment (data not presented). The same results were obtained using rabbit CK-M, and mouse brain extracts (data not presented).

Taken together, these results show that the native creatine kinases BB and MM are complexed with a morphine-type molecule. This association undoubtedly resulted from a complex with high affinity and gentle purification which was carried out in order to preserve the enzymatic activity of the creatine kinase.

### Effect of a Morphine Antagonist on Human Creatine Kinase BB and Immunoreactivity for the Alkaloids

In order to confirm that the morphine-type molecule is bound to creatine kinase BB, the protein (1 μg) was incubated or not with naloxone for 12 h at 37° C. The samples were deposited on polyacrylamide gel (urea buffer TP5, without heat treatment) and then transferred onto membrane which was subsequently detected with the 6D6 antibody.

The results show that the incubations with naloxone (not detected by the anti-morphine antibody 6D6) significantly decreased the alkaloid labeling (=morphine-type molecule) relative to the control experiments (the same incubation carried out in the absence of antagonist; FIG. 4D). In contrast, the immunoreactivity carried out in parallel on the same samples with the anti-CK-BB antibody is unchanged, as naloxone replaced the alkaloid initially bound (data not presented).

These results suggest that the morphine-type molecules are not bound covalently to creatine kinase BB, and that these alkaloids (morphine-type molecules) can be replaced with a morphine analog that is not detected by the anti-morphine antibody used. Identical results were observed for rabbit creatine kinase MM (data not presented).

Overall, the results show that creatine kinase B and creatine kinase M bind strongly, noncovalently, morphine and/or morphine derivatives possibly at the level of its C-terminal region, and that the presence of this ligand decreases the immunoreactivity for creatine kinase B and creatine kinase M (anti-C-terminal portion of creatine kinase antibody). However, we cannot rule out that other portions of creatine kinase also bind the alkaloids. In fact, it appears that the epitope recognized by the anti-creatine kinase antibody is partly masked by the bound alkaloids. Dissociation of the alkaloids releases the recognition site of the anti-creatine kinase antibody. Moreover, the experiments show that the loss of immunodetection for morphine and/or derivatives thereof is not dependent on degradation of the proteins, but is indeed due to dissociation of the alkaloids during a heat treatment in the presence of urea.

### LIST OF REFERENCES

1. Herbert et al., Nat. Prod. Rep., 17: 317-322, 2000
2. Stefano et al., Trends Neurosci., 23: 436-442, 2000]
3. Bianchi et al., Brain Res., 627: 210-215, 1993
4. Bianchi et al., Adv. Neuroimmunol., 4: 83-92, 1994
5. Guarna et al., J. Neurochem., 70: 147-152, 1998
6. Muller et al., PLoS One, 3: Epub www.plosone.org/doi/pone.0001641, 2008
7. Boettcher et al., Proc. Natl. Acad. Sci. USA, 102: 8495-8500, 2005
8. Goumon et al., An. R. Acad. Nac. Farm., 75: 389-418, 2009
9. Lotsch et Geisslinger, Clin. Pharmacokinet., 40: 485-499, 2001
10. Demande de Brevet en Europe No. 07301692.5
11. Laux et al., J. Comp. Neurol., 519(12): 2390-416, 2011
12. Charron et al., Brain, PMID: 21742735, 2011
13. Atmanene et al., Med. Sci. Monit., 15: BR178-187, 2009
14. Nagamatsu et hasegawa, Biochem. Pharmacol., 43: 2631-2635, 1992
15. Nagatmasu et Hasegawa, Drug Chem. Toxicol., 16: 241-253, 1993
16. Goumon et al., J. Biol. Chem., 281(12): 8082-8089, 2006
17. Peng et al. J. Proteome Res., 2(1): 43-50, 2003
18. Elias et Gygi, Nat. Methods, 4(3): 207-214, 2007
19. Van Dorp et al., Expert Opin. Drug Saf., 6(2): 125-32, 2007
20. Arun et al., Indian J. Med. Res., 107: 231-238, 1998
21. Glattard et al., PLoS One, 1: e8791, 2010
22. Goumon et al., Neurosci. Lett., 293: 135-138, 2000
23. Lee et Spector, J. Pharmacol. Exp. Ther., 257: 647-650, 1991
24. Madbouly et al., Br. J. Surg., 97: 759-764, 2010
25. Marrazzi et al., Life Sci., 60: 1741-1747, 1997
26. Batki et al., Am. J. Addict., 16(4): 253-259, 2007
27. Green et al., J. Subst. Abuse Treat., 34(1): 61-71, 2008
28. Goodman et al., ChemMedChem., 2(11): 1552-1570, 2007
29. Meyer, J. Clin. Psychopharmacol., 28(6): 722-723, 2008

1) A creatine kinase or derivative thereof for prevention or treatment of an addiction or of an addictive disorder associated with opiates.

2) The creatine kinase or derivative thereof as claimed in claim 1, wherein the opiates are natural opium alkaloids.

3) The creatine kinase or derivative thereof as claimed in claim 2, wherein the natural opium alkaloids are morphine and derivatives thereof.

4) The creatine kinase or derivative thereof as claimed in claim 1, wherein the opiates are endogenous natural alkaloids, preferably endogenous morphine and derivatives thereof.

5) A creatine kinase or derivative thereof for use as a medicinal product for capturing opiates.

6) A pharmaceutical composition for use in the prevention or treatment of an addiction or of an addictive disorder associated with opiates, comprising a creatine kinase or a derivative thereof as active principle and a pharmaceutically acceptable vehicle.

7) The pharmaceutical composition as claimed in claim 6, wherein the opiates are natural opium alkaloids.

8) The pharmaceutical composition as claimed in claim 7, wherein the natural opium alkaloids are morphine and derivatives thereof.

9) The pharmaceutical composition as claimed in claim 6, wherein the opiates are endogenous natural alkaloids, preferably endogenous morphine and derivatives thereof.

10) A method of analysis of opiates in a sample comprising:
a) contacting said sample with a creatine kinase or a derivative thereof;
b) detecting any complexes formed between said creatine kinase or said derivative thereof and at least one opiate.

11) The method of analysis as claimed in claim 10, said method further comprising a step c) in which the results of detection obtained in step b) are compared relative to a negative and/or positive control.

12) A method of qualitative and/or quantitative analysis of opiates in a sample comprising:
a) contacting said sample with a creatine kinase or a derivative thereof;
b) detecting any complexes formed between said creatine kinase or said derivative thereof and at least one opiate;
c) comparing the results of detection obtained in step b) against a standard curve.

13) The method of analysis as claimed in claim 10, wherein the opiates detected are selected from the exogenous or endogenous natural alkaloids, preferably morphine and derivatives thereof.

14) The method of analysis as claimed in claim 10, wherein said creatine kinase or said derivative thereof is labeled.

15) A kit for analysis of opiates comprising a creatine kinase or a derivative thereof, and means for detecting the complexes formed between said creatine kinase or said derivative thereof and at least one opiate.

16) The use of a creatine kinase or of a derivative thereof in an in vitro method of filtration of a body fluid for capturing opiates.

17) The use of a creatine kinase or of a derivative thereof as claimed in claim 16, wherein the body fluid is selected from urine, blood and cerebrospinal fluid.

18) A device for capturing opiates, characterized in that it comprises a system for dialysis of a body fluid in the presence of a creatine kinase or of a derivative thereof.

19) The method of analysis as claimed in claim 12, wherein the opiates detected are selected from the exogenous or endogenous natural alkaloids, preferably morphine and derivatives thereof.

20) The method of analysis as claimed claim 12, wherein said creatine kinase or said derivative thereof is labeled.
